(19) United States
(12) Patent Application Publication
(10) Pub. No.: US 2022/0208380 A1
(43) Pub. Date: Jun. 30, 2022

(54) MEDICAL CARE SUPPORT DEVICE, OPERATION METHOD AND OPERATION PROGRAM THEREOF, AND MEDICAL CARE SUPPORT SYSTEM

(71) Applicant: FUJIFILM Corporation, Tokyo (JP)

(72) Inventors: Tsuyoshi HIRAKAWA, Tokyo (JP); Hiroshi HIRAMATSU, Tokyo (JP); Keiji TSUBOTA, Tokyo (JP); Misaki KAWAHARA, Tokyo (JP)

(73) Assignee: FUJIFILM Corporation, Tokyo (JP)

(21) Appl. No.: 17/695,866

(22) Filed: Mar. 16, 2022

Related U.S. Application Data
(63) Continuation of application No. PCT/JP2020/030785, filed on Aug. 13, 2020.

(30) Foreign Application Priority Data
Sep. 27, 2019 (JP) 2019-177806

(51) Int. Cl. G16H 50/20; A61B 5/00

(52) U.S. Cl. CPC G16H 50/20; A61B 5/7267; A61B 5/742; A61B 5/7275

(57) ABSTRACT

Provided are a medical care support device, an operation method and a non-transitory computer readable recording medium storing an operation program thereof, and a medical care support system capable of promptly proposing necessary examinations and treatments in a case where a patient suffers from a chronic disease. A medical care support device (12) includes a display screen generation unit (62) and a prediction execution unit (63). The prediction execution unit (63) extracts a specific disease affecting a patient from acquired medical care information, and outputs a chronic disease and a complication that is likely to occur with a morbidity of the chronic disease as a prediction result. The display screen generation unit (62) proposes medical care practices for both the chronic disease and the complication.

### CROSS-REFERENCE TO RELATED APPLICATIONS

This application is a Continuation of PCT International Application No. PCT/JP2020/030785 filed on Aug. 13, 2020, which claims priority under 35 U.S.C. § 119(a) to Japanese Patent Application No. 2019-177806 filed on Sep. 27, 2019. Each of the above application(s) is hereby expressly incorporated by reference, in its entirety, into the present application.

### BACKGROUND OF THE INVENTION

### 1. Field of the Invention

The present invention relates to a medical care support device, an operation method and a non-transitory computer readable recording medium storing an operation program thereof, and a medical care support system.

### 2. Description of the Related Art

In the medical field, integrated medical care support devices and medical care support systems that share medical care processes and medical care results between medical staff or medical departments so that the medical staff such as doctors and laboratory technicians can smoothly proceed with medical examinations and tests are being used. The medical care support device supports medical care by providing the medical staff with, for example, displaying a list of medical care processes and medical care results for a plurality of patients (JP2016-143204A).

On the other hand, there is known a medical care support device having a function of estimating a patient's disease name from a patient's symptom diagnosed by a doctor (JP2008-27099A). In the medical care support device, for each combination of symptoms and disease names, diagnosis frequency information indicating the frequency in which the doctor has diagnosed a patient with the symptom as a disease with the disease name in the past is stored and the disease name of the patient is estimated based on the input diagnosis frequency information related to each symptom of the patient.

In recent years, the number of patients with chronic diseases represented by diabetes and hypertension, and the risk of developing chronic diseases have been increasing. As the proportion of elderly people suffering from chronic diseases increases, it is expected that the number will increase further in the present age when the population is aging all over the world including Japan.

### SUMMARY OF THE INVENTION

There is a shortage of specialists with advanced expertise for patients with chronic diseases that continue to increase with aging. In addition, in the opinion of patients, once a chronic disease develops, it is often necessary to continue treatment for the rest of their lives, but on the other hand, since there are few subjective symptoms of suffering from a chronic disease and it is difficult to feel the therapeutic effect, in the current situation, there are many cases in which necessary examinations and treatments are not received or interrupted in the middle of chronic diseases.

In contrast, in the medical care support device described in JP2016-143204A, the function of supporting the user in a case where the patient suffers from a chronic disease is not considered. Further, in the medical care support device described in JP2008-27099A, it cannot be estimated that the patient's disease name is a complication unless the information that the doctor has diagnosed as a complication in the past is stored.

Chronic diseases are difficult to cure once they progress, and in the case of diabetes, for example, once they progress, they lead to complications such as blindness, gangrene of limbs, and diabetic nephropathy, which can greatly reduce the quality of life of patients. There is a need for a medical care support device that can receive appropriate treatment before the progression of a chronic disease and can propose necessary examinations and treatments so as to delay the progression as much as possible.

Therefore, an object of the present invention is to provide a medical care support device, an operation method and a non-transitory computer readable recording medium storing an operation program thereof, and a medical care support system capable of promptly proposing necessary examinations and treatments in a case where a patient suffers from a chronic disease.

According to an aspect of the present invention, there is provided a medical care support device comprising a medical care information acquisition unit, a prediction execution unit, and a medical care practice proposal unit. The medical care information acquisition unit acquires medical care information of a patient from a terminal device installed in a medical facility or a server. The prediction execution unit acquires a specific disease affecting the patient from the acquired medical care information, and outputs the specific disease and a comorbidity that is likely to occur with a morbidity of the specific disease as a prediction result. The medical care practice proposal unit proposes medical care practices for both the specific disease and the comorbidity predicted by the prediction execution unit.

According to another aspect of the present invention, there is provided a medical care support device comprising a medical care information acquisition unit, and a medical care practice proposal unit. The medical care information acquisition unit acquires medical care information of a patient from a terminal device or a server installed in a medical facility. The medical care practice proposal unit proposes medical care practices for both a specific disease and a comorbidity that is likely to occur with a morbidity of the specific disease from the acquired medical care information by using a prediction result predicted by a prediction execution unit installed outside the medical facility, the prediction result including the specific disease affecting the patient and the comorbidity.

It is preferable that the prediction execution unit includes, as the prediction result, an examination or treatment to be performed for the specific disease and the comorbidity, and the medical care practice proposal unit displays, as the proposal, the examination or treatment on the terminal device.

It is preferable that the prediction execution unit includes, as the prediction result, a proposal time for performing an examination or treatment for the specific disease and the comorbidity, and the medical care practice proposal unit displays, as the proposal, a patient who has not yet undergone the examination or treatment at the proposal time on the terminal device.

It is preferable that the prediction execution unit predicts the specific disease affecting the patient from the medical care information, and predicts the comorbidity from the predicted specific disease.

It is preferable that the medical care support device further comprises: a user correction storage unit that, in a case where a user performs a different medical care practice with respect to the medical care practices proposed by the medical care practice proposal unit, accumulates the medical care practice performed by the user as a user correction content; and a prediction correction unit that outputs a prediction correction result obtained by correcting the prediction result using the accumulated user correction content, and the medical care practice proposal unit proposes a medical care practice in which the prediction correction result is reflected.

It is preferable that the prediction execution unit is configured by using a trained model that outputs a comorbidity that is likely to occur for a predetermined specific disease and medical care practices for both the specific disease and the comorbidity, and a new trained model is generated by performing machine learning using the accumulated user correction content as teacher data, and the trained model used for the prediction execution unit is updated.

It is preferable that the specific disease is a chronic disease, and the comorbidity is a complication that is likely to occur with a morbidity of the chronic disease.

It is preferable that the medical care practice includes the examination for the specific disease or the comorbidity.

It is preferable that the medical care practice includes medication for the patient with the specific disease.

According to another aspect of the present invention, there is provided a medical care support system comprising a medical care support device, a terminal device, and an external server.

According to another aspect of the present invention, there is provided an operation method of a medical care support device, the operation method comprising: a medical care information acquisition step of acquiring medical care information of a patient from a terminal device or a server installed in a medical facility; and a prediction execution step of extracting a specific disease affecting the patient from the acquired medical care information, and outputting the specific disease and a comorbidity that is likely to occur with a morbidity of the specific disease as a prediction result; and a medical care practice proposal step of proposing medical care practices for both the specific disease and the comorbidity predicted in the prediction execution step.

According to another aspect of the present invention, there is provided a non-transitory computer readable recording medium storing an operation program of a medical care support device, the operation program comprising: a medical care information acquisition step of acquiring medical care information of a patient from a terminal device or a server installed in a medical facility; and a prediction execution step of extracting a specific disease affecting the patient from the acquired medical care information, and outputting the specific disease and a comorbidity that is likely to occur with a morbidity of the specific disease as a prediction result; and a medical care practice proposal step of proposing medical care practices for both the specific disease and the comorbidity predicted in the prediction execution step.

According to the aspects of the present invention, in a case where a patient suffers from a chronic disease, necessary examinations and treatments can be promptly proposed.

### BRIEF DESCRIPTION OF THE DRAWINGS

FIG. 1 is an explanatory diagram showing a configuration of a medical care support system.

FIG. 2 is a block diagram showing a configuration of a client terminal.

FIG. 3 is a block diagram showing a function of the client terminal.

FIG. 4 is a block diagram showing a configuration of a medical care support device.

FIG. 5 is a block diagram showing a function of the medical care support device.

FIG. 6 is a block diagram showing a function of a prediction execution unit.

FIG. 7 is an explanatory diagram showing an example of a chronic disease, symptoms of each chronic disease, and complications that are likely to occur in the case of suffering from each chronic disease.

FIG. 8 is an explanatory diagram showing an example of complications that are likely to occur with diabetes and examinations necessary for each complication.

FIG. 9 is an explanatory diagram showing an example of complications that are likely to occur with hypertension and examinations necessary for each complication.

FIG. 10 is an explanatory diagram showing an example of complications that are likely to occur with dyslipidemia and examinations necessary for each complication.

FIG. 11 is an explanatory diagram showing an example of drugs to be administered for diabetes and complications that are likely to occur with diabetes.

FIG. 12 is an initial screen.

FIG. 13 is an example of a layout display screen in which a proposal is made by a medical care practice proposal unit.

FIG. 14 is an enlarged explanatory diagram of examination information as a proposal by the medical care practice proposal unit.

FIG. 15 is an explanatory diagram showing a configuration of a medical care support system according to a second embodiment.

FIG. 16 is a block diagram showing a configuration of a medical care support device and an external server according to the second embodiment.

FIG. 17 is a block diagram showing a configuration of a medical care support device and an external server according to a third embodiment.

FIG. 18 is a block diagram showing a configuration of a medical care support device and an external server according to a modification example of the third embodiment.

FIG. 19 is a flowchart illustrating processing of a proposal for a medical care practice according to a fourth embodiment.

FIG. 20 is an example of a display screen on which the medical care practice according to the fourth embodiment is proposed.

### DESCRIPTION OF THE PREFERRED EMBODIMENTS

### First Embodiment

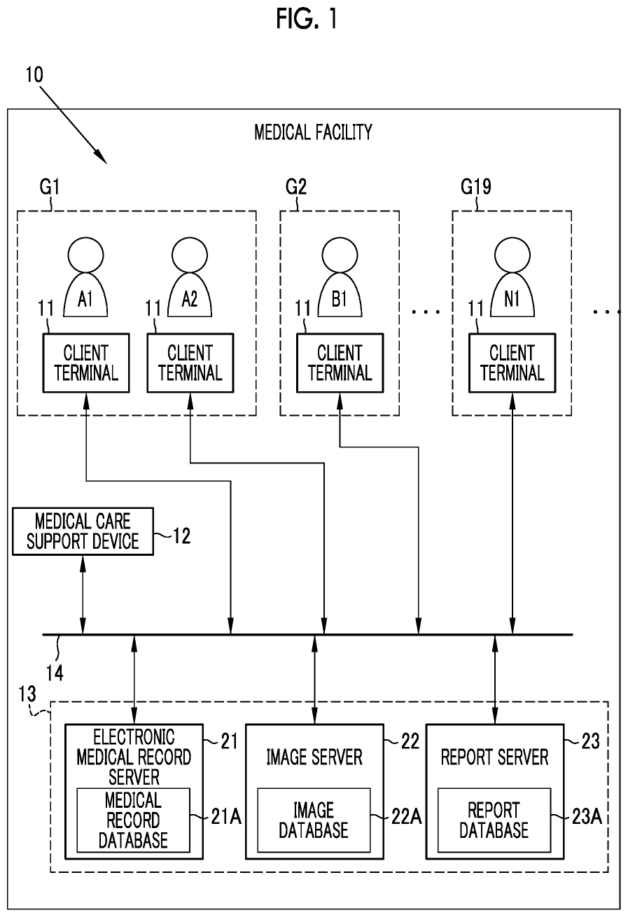

As shown in FIG. 1, a medical care support system 10 is a computer system that provides medical care support in a medical facility such as a hospital, and comprises a client terminal 11, a medical care support device 12, and a server group 13. These elements constituting the medical care support system 10 are connected to each other so as to be able to communicate with each other by using a network 14 such as a local area network (LAN) installed in the medical facility.

The client terminal 11 is a terminal for receiving a service (a function of the medical care support device 12) from the medical care support device 12, and is a computer directly operated by a medical staff such as a doctor, a laboratory technician, or a nurse (including the case of a tablet terminal, etc.), or the like. The client terminal 11 is installed in a medical department such as an internal medicine or a surgery, various examination departments such as a radiological examination department or a clinical examination department, a nurse center, or other necessary places. Further, the client terminal 11 can be provided for each medical staff, and can be shared by a plurality of medical staff. Therefore, the medical care support system 10 includes a plurality of client terminals 11. For example, a group G1 is an “internal medicine” to which a doctor A1 and a doctor A2 belong, and the doctor A1 and the doctor A2 each have a client terminal 11. Similarly, for example, a group G2 is a “surgery” to which a doctor B1 belongs, and the group G2 has at least one client terminal 11. Further, for example, a group G19 is a “radiology department” to which a technician N1 belongs, and the group G19 has at least one client terminal 11.

The medical care support device 12 provides the client terminal 11 with a display screen including medical care data (for example, an image or the like itself) and/or information indicating the location of the medical care data (for example, a link to an image or the like), for example, in response to a request from the client terminal 11. Medical care data is images, reports, examination results, and other medical care processes acquired or created in medical examinations, tests, surgeries, and the like, or is data obtained as a result of medical care or information indicating the location of these (so-called links (aliases), etc.). The medical care support device 12 acquires medical care data to be used on the display screen from the server group 13.

A display screen provided by the medical care support device 12 to the client terminal 11 refers to data used by the client terminal 11 to form a screen of a display unit 36 (see FIG. 3) of the client terminal 11. Further, the display screen provided by the medical care support device 12 to the client terminal 11 includes not only data for full-screen display that the client terminal 11 constitutes the display of the entire screen but also data that constitutes the display related to a part of the screen. For example, in the present embodiment, the medical care support device 12 provides the client terminal 11 with a display screen that can be displayed in a general window format on a part of the screen of the display unit 36.

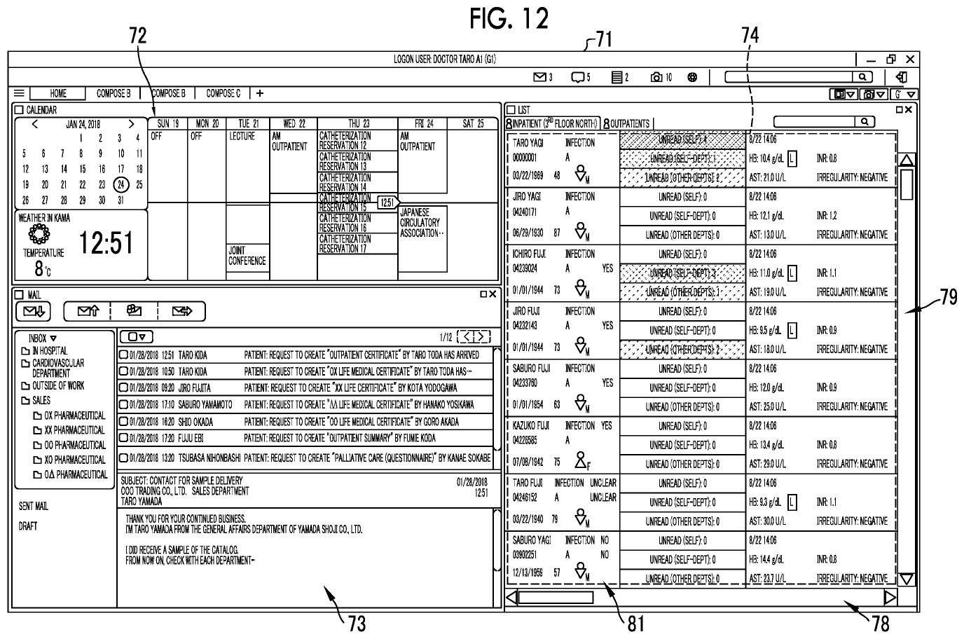

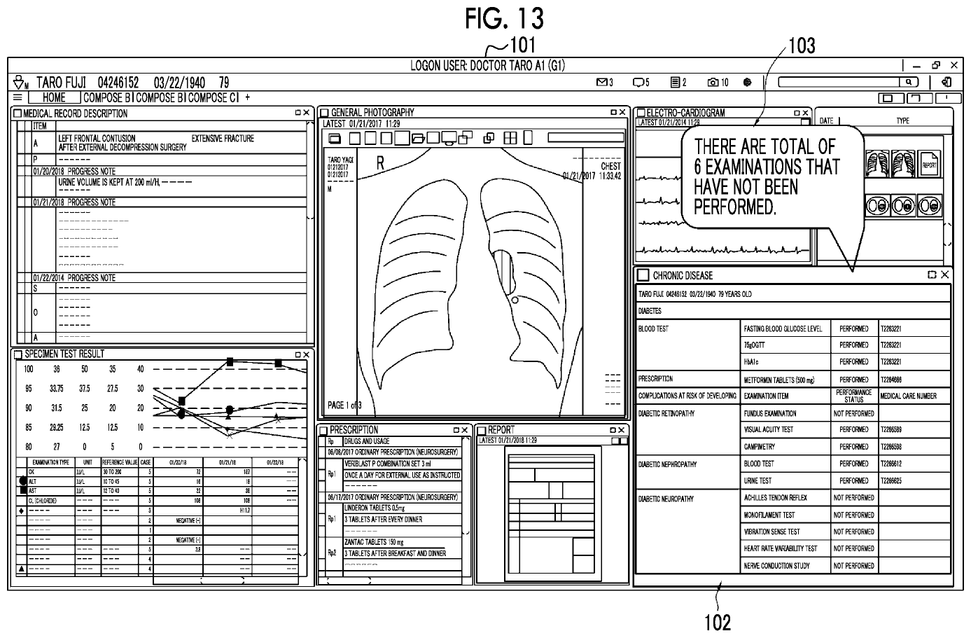

Specifically, the display screens provided by the medical care support device 12 to the client terminal 11 include a clinical flow screen 81 (see FIG. 12), a timeline screen (not shown), a layout display screen 101 (see FIG. 13), and the like. The clinical flow screen 81 is a display screen for displaying patient identification information and a part or all of the medical care process in association with each other for each of a plurality of patients. Patient identification information is, for example, identification data (ID) such as the patient's name, date of birth, age, or gender, or a unique number and/or symbol given to the patient (hereinafter referred to as a patient ID). A medical care process refers to the process or result of medical care that has already been performed and that is scheduled to be performed in the future. Therefore, the medical care process may include not only medical care data that has already been acquired, but also medical care data that is scheduled to be acquired. The medical care data that is scheduled to be acquired is, for example, information regarding the presence or absence of an order for a specific examination, a scheduled date and time thereof, the type of medical care data that is scheduled to be acquired, and the like. Further, in the present embodiment, in a case where one medical care process includes a plurality of items (items such as examination results), the term “medical care process” refers to any of the items that constitute the medical care process, not the entire medical care process (a collection of a plurality of items). The timeline screen is a display screen for displaying a part or all of the medical care process of a specific patient on one screen in a time series. The layout display screen 101 is a display screen for displaying a part or all of the medical care process of a specific patient by arranging them vertically and horizontally (for example, arranging them in a tile shape).

The medical care support device 12 provides a display screen to the client terminal 11 in a description format using a markup language such as extensible markup language (XML) data, for example. The client terminal 11 displays an XML format display screen using a web browser. The medical care support device 12 can provide a display screen to the client terminal 11 in another format such as JavaScript (registered trademark) object notation (JSON) instead of XML.

The server group 13 searches for medical care data in response to the request from the medical care support device 12, and provides the medical care data corresponding to the request to the medical care support device 12. The server group 13 includes an electronic medical record server 21, an image server 22, a report server 23, and the like.

The electronic medical record server 21 has a medical record database 21A for storing electronic medical records. An electronic medical record is a collection of one or a plurality of pieces of medical care data. Specifically, the electronic medical record includes, for example, medical care data such as a medical examination record, a result of a specimen test, a patient's vital sign, an order for tests, a treatment record, or accounting data. The electronic medical record can be input and viewed using the client terminal 11.

A medical examination record is a record of the contents and results of the interview or palpation, the disease name, or the like. A specimen is blood or tissue collected from a patient, or the like, and a specimen test is a blood test, a biochemical test, or the like. A vital sign is data indicating a patient's condition such as a patient's pulse, blood pressure, or body temperature. An order for examinations is a request for examinations such as a specimen test, photography using various modality, report creation, treatment or surgery, medication, or the like. A treatment record is a record of treatment, surgery, medication, prescription, or the like. Accounting data is data related to consultation fees, drug fees, hospitalization fees, and the like.

The image server 22 is a so-called PACS (picture archiving and communication system) server, and has an image database 22A in which examination images are stored. An examination image is an image obtained by various image examinations such as computed tomography (CT) examination, magnetic resonance imaging (MRI) examination, X-ray examination, ultrasonography, and endoscopy. These examination images are recorded in a format conforming to, for example, the digital imaging and communications in medicine (DICOM) standard. The examination image can be viewed using the client terminal 11.

The report server 23 has a report database 23A for storing an interpretation report. An interpretation report (hereinafter simply referred to as a report) is a report that summarizes the interpretation results of the examination image obtained by the image examination. The interpretation of the examination image is performed by a radiologist. The report can be created and/or viewed using the client terminal 11.

A patient ID is attached to each of the above electronic medical records, examination images, and reports. In addition to the patient ID, information that identifies the medical staff who input the medical care data for each piece of medical care data is attached to the electronic medical record. In addition to the patient ID, information that identifies the medical staff (specifically, the laboratory technician) who performed the examination is attached to the examination image. Information that identifies the medical staff (specifically, the radiologist) that created the report is attached to the report. Information that identifies the medical staff is an ID such as the name of the medical staff or a unique number and/or symbol given to each medical staff (hereinafter referred to as a medical staff ID).

The client terminal 11, the medical care support device 12, and the servers 21 to 23 constituting the server group 13 are configured by installing an operating system program and an application program such as a server program or a client program based on a computer such as a server computer, a personal computer, or a workstation. That is, the basic configurations of the client terminal 11, the medical care support device 12, and the servers 21 to 23 constituting the server group 13 are the same, and a central processing unit (CPU), a memory, a storage, a communication unit, etc., and a connection circuit for connecting these are provided. The communication unit is a communication interface (LAN interface board or the like) for connecting to the network 14. The connection circuit is, for example, a motherboard that provides a system bus and/or a data bus and the like.

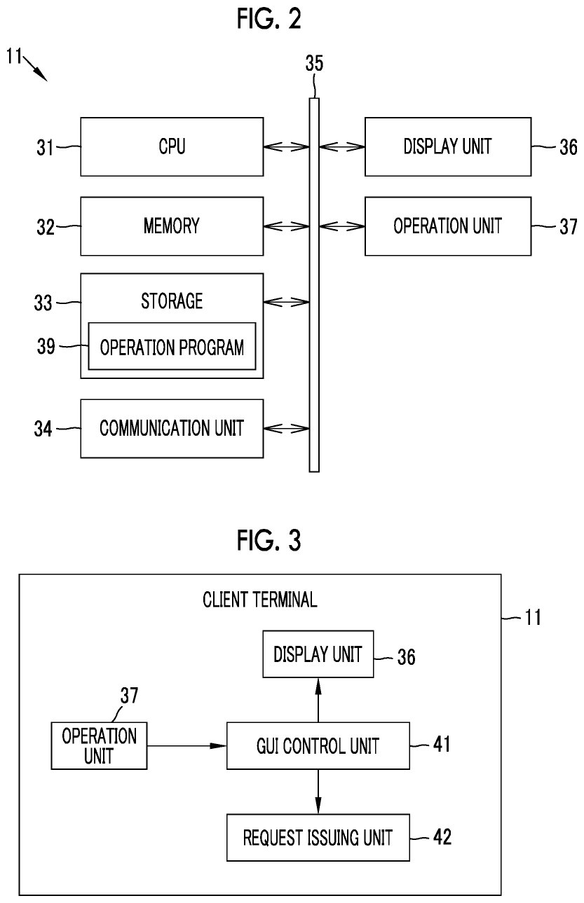

As shown in FIG. 2, the client terminal 11 comprises a display unit 36 and an operation unit 37 in addition to a CPU 31, a memory 32, a storage 33, a communication unit 34, and a connection circuit 35. The display unit 36 is, for example, a display using a liquid crystal display or the like, and has at least a screen for displaying a display screen provided by the medical care support device 12. The operation unit 37 is, for example, a pointing device such as a mouse and/or an input device such as a keyboard. The display unit 36 and the operation unit 37 can form a so-called touch panel.

The client terminal 11 stores an operation program 39 in addition to the operating system program and the like in the storage 33. The operation program 39 is an application program for receiving the function of the medical care support device 12 by using the client terminal 11. In the present embodiment, the operation program 39 is a web browser program. Here, the operation program 39 can be a dedicated application program for receiving the function of the medical care support device 12. The operation program 39 may include one or a plurality of gadget engines for controlling a part or all of the display screen provided by the medical care support device 12. A gadget engine is a subprogram that exhibits various functions by operating alongside a web browser or the like.

In a case where the operation program 39 is activated in the client terminal 11, as shown in FIG. 3, the CPU 31 of the client terminal 11 functions as a graphical user interface (GUI) control unit 41 and a request issuing unit 42 in cooperation with the memory 32.

The GUI control unit 41 displays the display screen provided by the medical care support device 12 on the web browser in the display unit 36. The GUI control unit 41 controls the client terminal 11 in response to an operation instruction input using the operation unit 37, such as a button click operation with a pointer.

The request issuing unit 42 issues various processing requests (hereinafter referred to as processing requests) to the medical care support device 12 in response to the operation instruction of the operation unit 37. The processing request issued by the request issuing unit 42 is, for example, a distribution request for the display screen, an edit request for the display screen, or the like. The request issuing unit 42 transmits the processing request to the medical care support device 12 via the communication unit 34 and the network 14.

The distribution request for the display screen is to request the medical care support device 12 to distribute a display screen having a specific configuration. For example, the distribution can be received by designating any one of the clinical flow screen 81, the timeline screen, the layout display screen 101, and the like, depending on the distribution request for the display screen.

The edit request for the display screen is to request the medical care support device 12 to edit the contents of the medical care data and the like to be displayed on the display screen after receiving the distribution of the display screen having a specific configuration from the medical care support device 12. For example, in a case where the distribution of the clinical flow screen 81 is received, the edit request for the display screen is a request for designating or changing a list of patients to be displayed, designating or changing a display target period of the medical care process, designating or changing the medical care process to be displayed, or sorting the display contents.

The distribution request and/or edit request for the display screen includes information such as a medical staff ID and an address on the network of the client terminal 11. The medical staff ID is entered on a login screen (not shown) for the medical care support system 10 (or the medical care support device 12).

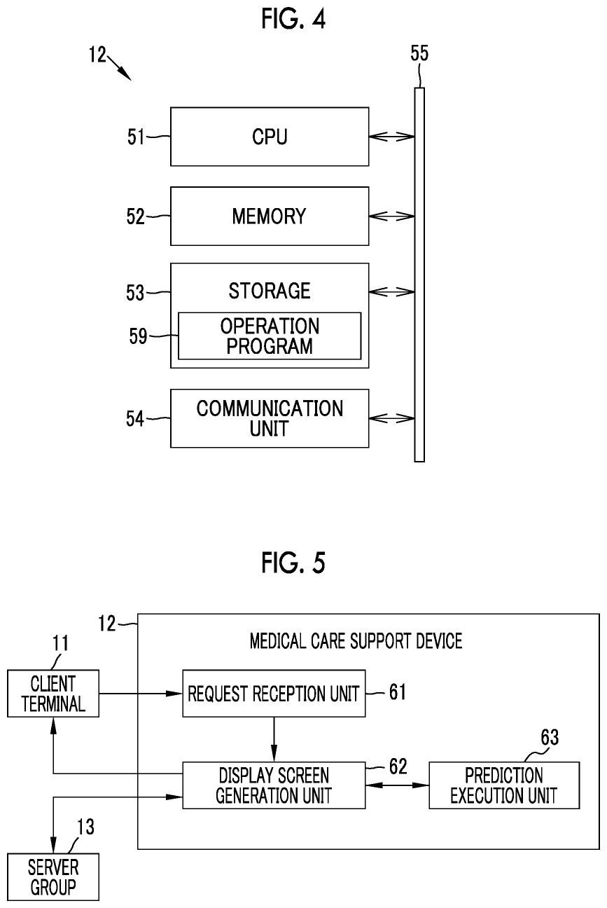

As shown in FIG. 4, the medical care support device 12 comprises a CPU 51, a memory 52, a storage 53, a communication unit 54, and a connection circuit 55. The medical care support device 12 can comprise a display unit and/or an operation unit as necessary, like the client terminal 11, and can be attached with a display unit and/or an operation unit as necessary, but in the present embodiment, the medical care support device 12 does not have a display unit and an operation unit.

The medical care support device 12 stores an operation program 59 in addition to the operating system and the like in the storage 53. The operation program 59 is an application program for causing the computer constituting the medical care support device 12 to function as the medical care support device 12. In a case where the operation program 59 is activated, as shown in FIG. 5, the CPU 51 of the medical care support device 12 functions as a request reception unit 61, a display screen generation unit 62, a prediction execution unit 63, and the like in cooperation with the memory 52.

The request reception unit 61 receives various processing requests such as a distribution request and an edit request for the display screen from the client terminal 11. In a case where the request reception unit 61 receives various processing requests, the request reception unit 61 inputs a processing instruction to each unit that executes the corresponding processing according to the content of the requested processing. For example, in a case where there is a distribution request for the display screen from the client terminal 11, the request reception unit 61 inputs a generation instruction of the corresponding display screen to the display screen generation unit 62. Similarly, in a case where there is an edit request for the display screen from the client terminal 11, the request reception unit 61 inputs an edit instruction of the corresponding display screen to the display screen generation unit 62. The request reception unit 61 also receives a request to log in to the medical care support device 12, and a login processing unit (not shown) executes login processing such as confirmation of the medical staff ID and password.

The display screen generation unit 62 generates or edits various display screens such as the layout display screen 101. In the present embodiment, in a case where there is a new distribution request for the display screen, the display screen generation unit 62 generates XML data representing the display screen, and in a case where there is an edit request for the display screen, the display screen generation unit 62 edits the XML data created earlier according to the request content. The display screen generation unit 62 accesses the server group 13 as necessary, and acquires information regarding a medical care process or the like used for generating or editing the display screen. The display screen generation unit 62 can hold a part or all of the information regarding the medical care process or the like acquired from the server group 13 in order to reduce the access frequency to the server group 13. In a case where the login processing unit normally completes the login processing, the display screen generation unit 62 generates an initial screen 71 (see FIG. 12) to be displayed first after login. Further, in the case of creating or editing the initial screen 71, the display screen generation unit 62 acquires the information necessary for generating or editing the initial screen 71 from the server group 13, the client terminal 11, or another device or system that is linked with the medical care support system 10.

The prediction execution unit 63 also functions as a medical care information acquisition unit, and acquires medical care information of a patient among the information used by the display screen generation unit 62 to generate the display screen (medical care information acquisition step). Specifically, in a case where the display screen is generated, the display screen generation unit 62 acquires medical care information of a patient from the electronic medical record, the examination image, and the report acquired from the server group 13.

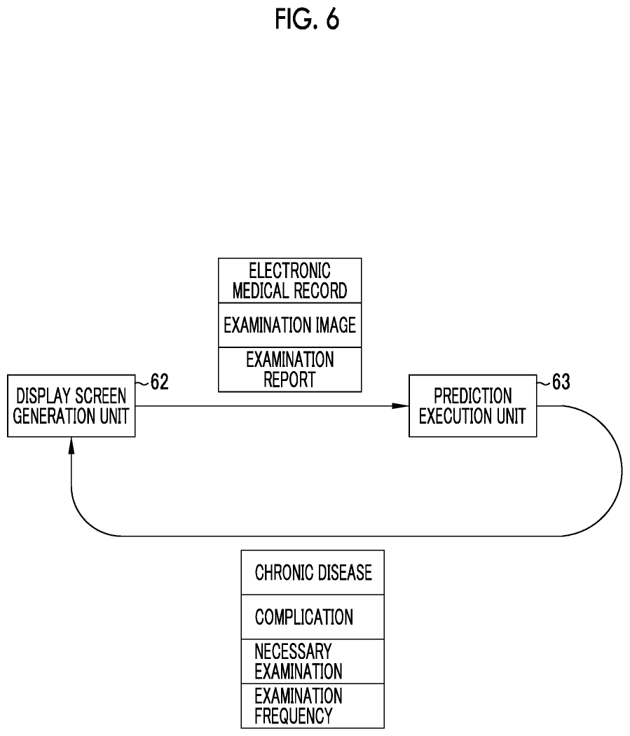

As shown in FIG. 6, the prediction execution unit 63 acquires medical care information of a patient from the electronic medical record, the examination image, and the report, extracts a chronic disease affecting the patient from the acquired medical care information, and predicts a complication that is likely to occur with a morbidity of the chronic disease. Further, in the present embodiment, in addition to the above-mentioned chronic disease and the complication that is likely to occur with the morbidity of the chronic disease, examinations necessary for each complication, the frequency of necessary examinations to receive the examinations, and drugs to be administered for chronic diseases and complications are also predicted.

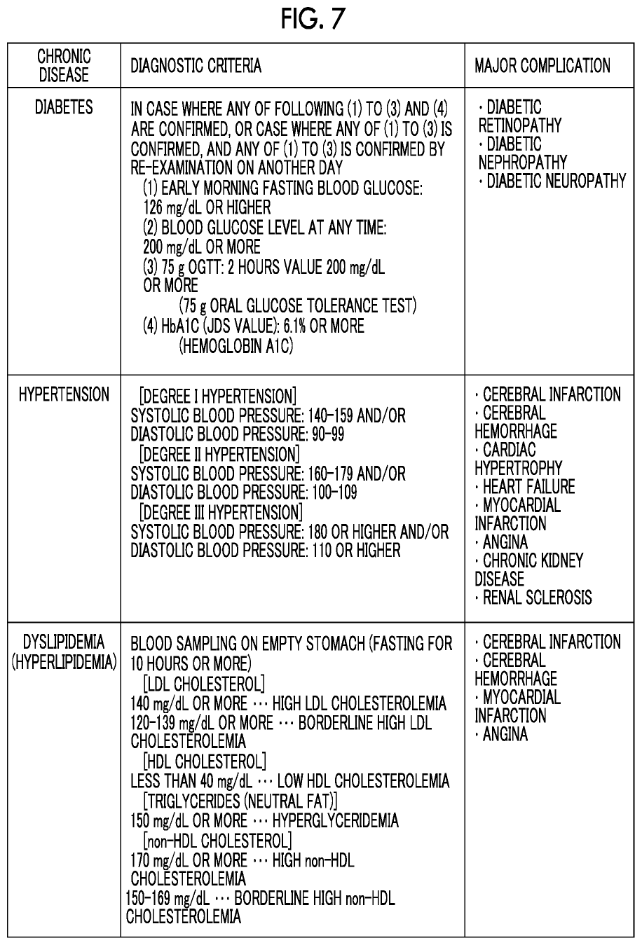

The prediction execution unit 63 is configured by using a trained model (so-called artificial intelligence (AI) program) generated by machine learning. The trained model uses data as shown in FIGS. 7 to 11 for machine learning as teacher data for predicting complications. Such data uses literature, papers, reports, etc. published by research institutes, medical institutions, academic organizations, etc. that are studying each chronic disease, and is collected in advance as big data on the web, or is input to a learning device as text data for learning. A manufacturer holds the machine-learned trained model as a program and writes it in a storage area for program storage (memory, storage, etc.) in the medical care support device 12 at the time of manufacturing the medical care support device 12 or performing a version update of the software.

FIG. 7 shows chronic diseases such as diabetes, hypertension, and dyslipidemia (hyperlipidemia), symptoms of each chronic disease, and complications that are likely to occur in the case of suffering from each chronic disease. For example, in a case where a person suffers from a chronic disease called diabetes, there is a high possibility of complications such as diabetic retinopathy, diabetic nephropathy, and diabetic neuropathy.

In addition, the chronic disease of hypertension has a difference in the degree of progress from degree I to degree III, and as the degree of progress progresses, the number of complications increases and the possibility of each complication also increases. Regarding dyslipidemia, there are differences in the degree of progress and types of cholesterol, such as low LDL (Low Density Lipoprotein) cholesterolemia, borderline high LDL cholesterolemia, low HDL (High Density Lipoprotein) cholesterolemia, hyperglyceridemia, high non-HDL cholesterolemia, and borderline high non-HDL cholesterolemia, and due to these differences, the types of complications and the possibility of each complication differ.

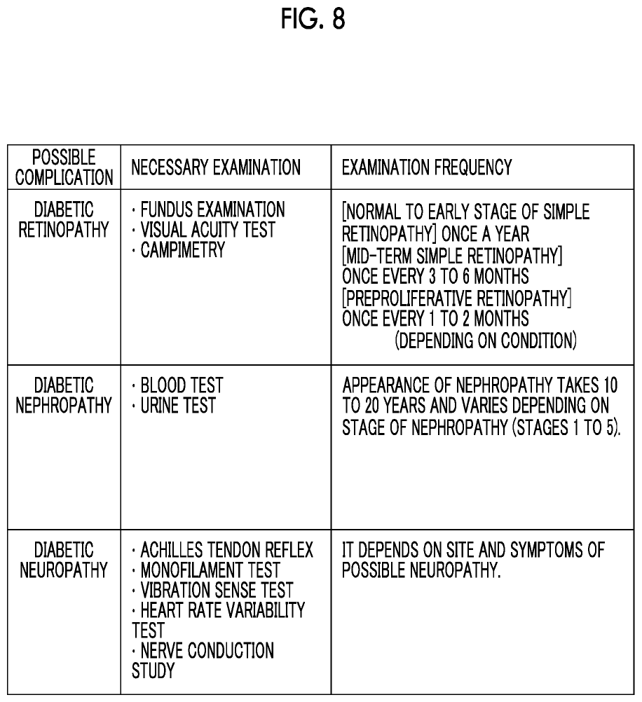

FIG. 8 is data on complications that are likely to occur with a chronic disease called diabetes, examinations required for each complication, and the frequency of necessary examinations in the case of undergoing the examinations. For example, in the case of diabetic retinopathy as a complication, the examinations necessary for diabetic retinopathy are fundus examination, visual acuity test, and campimetry, and indicates an examination frequency of once a year, once every 3 to 6 months, or once every 1 to 2 months, depending on the degree of progress of diabetic retinopathy.

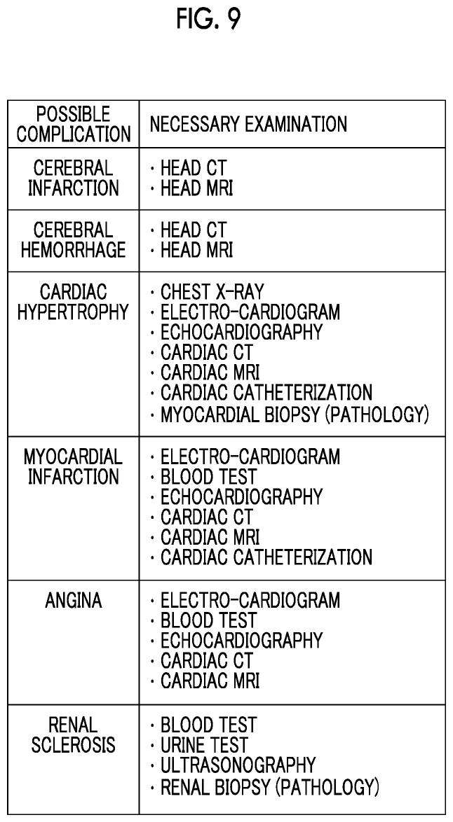

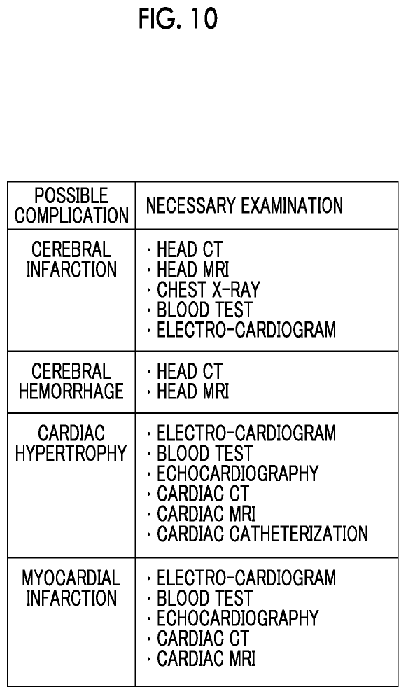

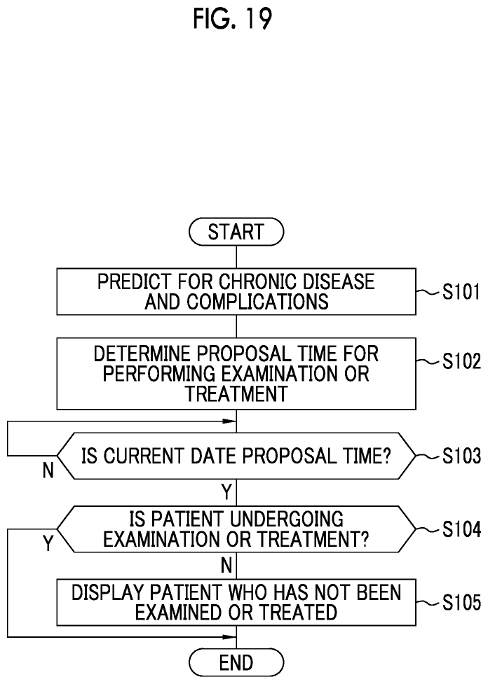

FIG. 9 shows data on complications that are likely to occur with a chronic disease called hypertension and examinations necessary for each complication, and FIG. 10 shows complications that are likely to occur with a chronic disease called dyslipidemia, and necessary examinations for each complication. Further, although omitted in FIGS. 9 and 19, data on the examination frequency for examinations necessary for hypertension and dyslipidemia are also used.

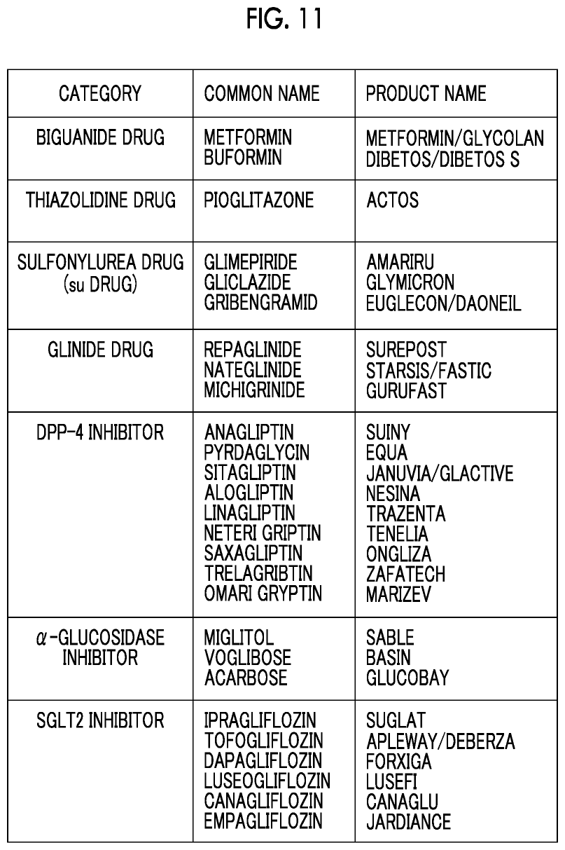

FIG. 11 is an example of drugs to be administered for diabetes and complications that are likely to occur with diabetes. For diabetes, there are seven types of drugs: biguanide drug, thiazolidine drug, sulfonylurea drug, glinide drug, DPP-4 inhibitor, α-glucosidase inhibitor, and SGLT2 inhibitor, and there is one or more types of drugs in each line of drugs, and there are a total of 28 types of drugs. In addition, the drugs include common names that are often used at the time of prescribing by doctors and product names that correspond to them. Further, although omitted in FIG. 11, for these drugs, combinations that can be used in combination and contraindicated data (drugs that should not be used for patients with certain symptoms or combinations of drugs that should not be used in combination) are also used as teacher data for machine learning. Contraindicated data is acquired, for example, from the regulatory package insert attached to the drug sold by each manufacturer.

The prediction execution unit 63 outputs a prediction result of predicting a chronic disease, complications, and the like from medical care information to the display screen generation unit 62 by using the above-mentioned trained model. The operation of outputting the prediction result of predicting a chronic disease, complications, and the like from the medical care information constitutes a prediction execution step.

In the present embodiment, the display screen generation unit 62 proposes medical care practices to the client terminal 11 for both the chronic disease and complications that the patient has suffered based on the prediction result predicted by the prediction execution unit 63. Specifically, the display screen generation unit 62 generates or edits XML data representing the display screen by using the prediction result of the chronic disease, complications, and the like predicted by the prediction execution unit 63, and transmits the XML data to the client terminal 11. The operation in which the display screen generation unit 62 makes a proposal to the client terminal 11 based on the prediction result predicted by the prediction execution unit 63 constitutes a medical care practice proposal step.

The medical care support system 10 configured as described above operates as follows. First, in a case where the medical staff logs in to the medical care support system 10 using the client terminal 11, the display screen generation unit 62 generates the initial screen 71 shown in FIG. 12 based on the settings and the like set for each medical staff, and provides the initial screen 71 to the client terminal 11. Thereby, the client terminal 11 displays the initial screen 71 on the screen of the display unit 36.

The initial screen 71 has, for example, three display fields of a schedule display field 72, a mail display field 73, and a list display field 74. The display contents of the schedule display field 72 and the mail display field 73 are generated by a gadget engine, which is a part of the operation program 39 of the client terminal 11, by obtaining information from the client terminal 11 or other devices or systems. Further, in the present embodiment, the list display field 74 displays at least a part of the clinical flow screen 81. Therefore, the display screen generation unit 62 generates the initial screen 71 including the schedule display field 72 and the mail display field 73 that do not include the contents, and the list display field 74 that includes the contents of the clinical flow screen 81. The client terminal 11 uses a gadget engine to display the initial screen 71 supplemented with the contents of the schedule display field 72 and the mail display field 73 on the screen of the display unit 36.

In a case where all the contents to be displayed do not fit in the list display field 74, a scroll bar 78 and a scroll bar 79 for transitioning (so-called scrolling) the display contents of the list display field 74 are displayed in the list display field 74 or in the vicinity of the list display field 74. The scroll bar 78 is a GUI that is operated in a case where the display content of the list display field 74 is changed in the horizontal direction and anon-display portion is displayed. The scroll bar 79 is a GUI that is operated in a case where the display content of the list display field 74 is changed in the vertical direction and a non-display portion is displayed. The GUI control unit 41 performs such GUI display and control.

On the above initial screen 71, for example, in a case where a predetermined menu or the like is operated using a GUI such as a pointer (not shown), the request issuing unit 42 issues a distribution request for the display screen. In the present embodiment, in order to display the layout display screen 101 that is not displayed on the initial screen 71, an operation for displaying the layout display screen 101, for example, an input operation for selecting one of the patients displayed in the list display field 74 is executed by using the GUI. Thereby, the request issuing unit 42 issues a distribution request for the layout display screen 101.

In a case where the request issuing unit 42 issues a distribution request for the display screen, in the medical care support device 12, the request reception unit 61 receives the distribution request for the display screen, and the display screen generation unit 62 generates the display screen related to the distribution request for the display screen. In the present embodiment, the display screen generation unit 62 refers to the patient identification information (for example, the patient ID) included in the list display field 74, and acquires the information related to the patient. Specifically, an electronic medical record, an examination image, a report, and the like to which the same patient identification information as the patient identification information included in the list display field 74 is attached are appropriately acquired from the server group 13 or the like. Then, the layout display screen 101 is generated by using the information related to the patient acquired by referring to the patient identification information.

As described above, in a case where the display screen generation unit 62 generates the display screen related to the distribution request, before the generation of the display screen, at the same time as the generation of the display screen (in parallel with the generation of the display screen), or after the display screen is generated, the prediction execution unit 63 outputs a prediction result regarding complications and the like to the medical care information of the patient. That is, the prediction execution unit 63 performs an input operation for selecting one patient on the client terminal 11, acquires medical care information from information such as an electronic medical record, an examination image, and a report of one patient acquired to create a layout display screen 101 (see FIG. 12) for this input operation, extracts the chronic disease affecting the patient from the acquired medical care information, and outputs the complications that are likely to occur with a morbidity of the chronic disease as a prediction result.

In the example shown in FIG. 13, one patient selected by the user's input operation on the client terminal 11 is “Taro Fuji”. Therefore, the prediction execution unit 63 acquires medical care information from the information such as the electronic medical record, the examination image, and the report with the patient ID of “Taro Fuji”, extracts a chronic disease affecting “Taro Fuji” from the acquired medical care information, and predicts a complication that is likely to occur with a morbidity of the chronic disease. In this case, the chronic disease of “Taro Fuji” is diabetes, and the complications that are likely to occur are “diabetic retinopathy”, “diabetic nephropathy”, and “diabetic neuropathy”. In addition, the prediction execution unit 63 may also output information on the examinations necessary for these complications as a prediction result.

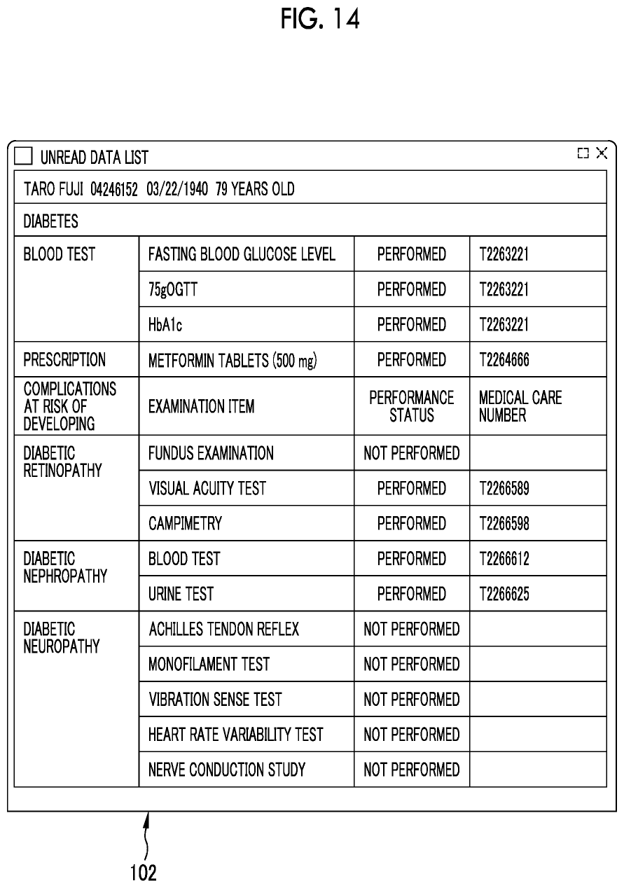

Next, the display screen generation unit 62 makes a proposal to the client terminal 11 based on the prediction result predicted by the prediction execution unit 63. That is, based on the prediction results of a chronic disease, complications, and the like predicted by the prediction execution unit 63, as shown in FIGS. 13 and 14, the display screen in which the layout display screen 101 is superimposed and displayed with the examination information 102 regarding the examinations and medications to be performed for both the chronic diseases and the complications is edited.

In this case, the example of the examination information 102 superimposed and displayed on the layout display screen 101 includes examination information regarding “diabetes”, which is a chronic disease that is likely to affect “Taro Fuji”, “diabetic retinopathy”, “diabetic nephropathy”, and “diabetic neuropathy”, which are complications that are likely to occur in the case of “diabetes”. Further, in the examples shown in FIGS. 13 and 14, information that the examinations necessary for “diabetes”, “diabetic retinopathy”, “diabetic nephropathy”, and “diabetic neuropathy” have been performed or have not been performed, and information on “metformin tablets (500 mg)”, which is a drug to be administered for “diabetes”, are also displayed. In addition, a display 103 is also displayed in the vicinity of the examination information 102 to call attention to “there are a total of 6 examinations that have not been performed.”

After that, the GUI control unit 41 of the client terminal 11 receives the distribution of the display screen edited as described above, and the distributed screen is displayed on the screen of the display unit 36 instead of the initial screen 71 initially displayed.

As described above, in the medical care support system 10 and the medical care support device 12 of the present embodiment, the chronic disease affecting the patient is extracted from the medical care information, complications that are likely to occur with a morbidity of the chronic disease are predicted, and examination information regarding the predicted chronic disease and complications is displayed on the client terminal 11. Therefore, in a case where a patient suffers from a chronic disease, proposals are made not only for the affected chronic diseases but also for complications that are likely to occur, and necessary examinations and treatments can be promptly proposed to patients.

### Second Embodiment

In the first embodiment, the prediction execution unit 63 provided in the medical care support device 12 extracts the chronic disease affecting the patient, and predicts the complications that are likely to occur with a morbidity of the chronic disease. However, the present invention is not limited thereto, and the prediction execution unit may be provided on an external server provided outside the medical facility to perform the prediction.

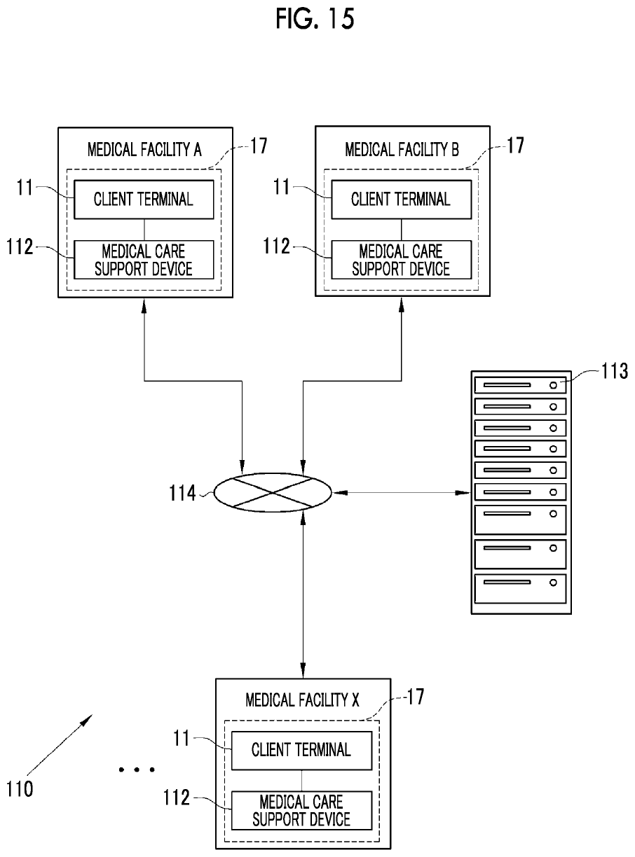

As shown in FIG. 15, a medical care support system 110 is a computer system that provides medical care support in a medical facility such as a hospital, and comprises medical care support devices 112 installed in a plurality of medical facilities A, B, . . . , X, client terminals 11 installed in the same medical facilities A, B, . . . , X as the medical care support device 112, an external server 113, a network 114, and the like. Although not shown in detail, each of the medical facilities A, B, . . . , X is provided with a server group 13 and a network 14 similar to those in the first embodiment, and the medical care support system 110 also includes the server group 13 and the network 14 provided in each of the medical facilities A, B, . . . , X. Further, a plurality of medical care support devices 112 may be installed in each of the medical facilities A, B, . . . , X. The external server 113 is an external server installed on the cloud. Further, the same devices or configurations as those of the first embodiment are designated by the same reference numerals and the description thereof will be omitted.

The network 114 is a wide area network (WAN) that widely connects the medical care support device 112 placed in the plurality of medical facilities A, B, . . . , X and the external server 113 via a public line network such as the Internet or a dedicated line network.

The client terminal 11 requests various processes from the medical care support device 112 as in the first embodiment, and displays the distributed display screen. As in the first embodiment, the server group 13 searches for medical care data in response to the request from the medical care support device 112, and provides the medical care data corresponding to the request to the medical care support device 112.

The basic configuration of the medical care support device 112 and the external server 113 is the same as that of the medical care support device 12 of the first embodiment, and is a high-performance computer having a well-known hardware configuration such as the CPU 51, the memory 52, the storage 53, the communication unit 54, and the connection circuit 55, and a well-known operation system and the like installed therein, and further having a server function.

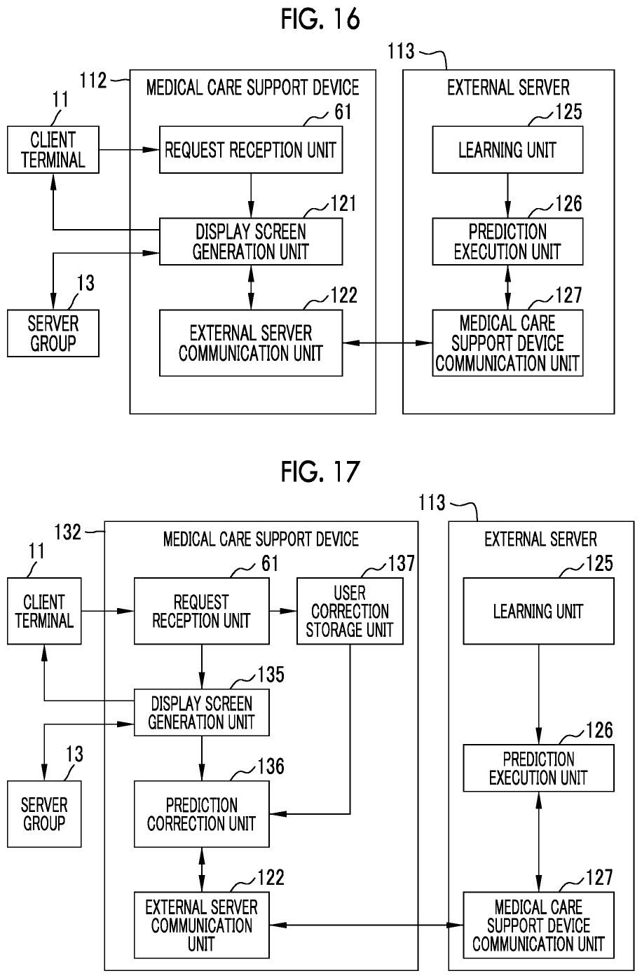

As shown in FIG. 16, the CPU 51 of the medical care support device 112 functions as the request reception unit 61, a display screen generation unit 121, an external server communication unit 122, and the like in cooperation with the memory 52.

The display screen generation unit 121 generates or edits various display screens in the same manner as the display screen generation unit 62 of the first embodiment. The display screen generation unit 121 proposes a medical care practice to the client terminal 11 based on a prediction result predicted by a prediction execution unit 126, which will be described later. The display screen generation unit 121 generates or edits XML data representing the display screen by using the prediction result of the chronic disease, complications, and the like predicted by the prediction execution unit 126, and transmits the XML data to the client terminal 11. The operation in which the display screen generation unit 121 makes a proposal to the client terminal 11 based on the prediction result predicted by the prediction execution unit 126 constitutes a medical care practice proposal step.

The external server communication unit 122 communicates with the external server 113 via the network 114. The external server communication unit 122 transmits the medical care information of the patient among the information used by the display screen generation unit 62 to generate the display screen, and receives the prediction result predicted by the prediction execution unit 126.

The CPU 51 of the external server 113 functions as a learning unit 125, the prediction execution unit 126, a medical care support device communication unit 127, and the like in cooperation with the memory 52.

The medical care support device communication unit 127 communicates with the medical care support device 112 via the network 114. The medical care support device communication unit 127 receives the medical care information of the patient, and transmits the prediction result predicted by the prediction execution unit 126.

The learning unit 125 generates a trained model used by the prediction execution unit 126. The trained model is generated by machine learning the same data as the teacher data described in the first embodiment. Such data uses literature, papers, reports, etc. published by research institutes, medical institutions, academic organizations, etc. that are studying each chronic disease, and is collected in advance as big data on the web, or is input to the learning unit 125 as text data for learning.

The trained model generated by the learning unit 125 is output to the prediction execution unit 126. Specifically, in a case where a trained model is generated, the trained model used for the prediction execution unit 126 may be constantly updated to the latest version, or the trained model may be updated periodically, such as the case where a version update of the software of the prediction execution unit 126 is performed.

The prediction execution unit 126 acquires the patient's medical care information via the external server communication unit 122, the network 114, and the medical care support device communication unit 127. The prediction execution unit 126 outputs the prediction result of predicting a chronic disease, complications, and the like from the acquired medical care information by using the above-mentioned trained model. The prediction result output by the prediction execution unit 126 is output to the display screen generation unit 121 via the medical care support device communication unit 127, the network 114, and the external server communication unit 122. The operation of outputting the prediction result of predicting a chronic disease, complications, and the like from the medical care information constitutes a prediction execution step.

The medical care support system 110 configured as described above operates as follows. Note that, the process is the same as in the first embodiment from the time when the medical staff logs in to the medical care support system 110 using the client terminal 11 until the request reception unit 61 receives the distribution request for the display screen, and the description thereof will be omitted.

The display screen generation unit 121 refers to patient identification information (for example, patient ID) for one patient selected by the client terminal 11, and acquires information related to the patient. The layout display screen 101 is generated by using the information related to the patient acquired by referring to the patient identification information.

In a case where the display screen generation unit 121 generates the display screen related to the distribution request, before the generation of the display screen, at the same time as the generation of the display screen (in parallel with the generation of the display screen), or after the display screen is generated, the external server communication unit 122 output medical care information of the patient.

The prediction execution unit 126 outputs the prediction result of predicting a chronic disease, complications, and the like from the acquired medical care information. The prediction result output by the prediction execution unit 126 is output to the display screen generation unit 121 via the medical care support device communication unit 127, the network 114, and the external server communication unit 122.

Next, based on the prediction result predicted by the prediction execution unit 126, the display screen generation unit 121 edits the display screen in which the layout display screen 101 is superimposed and displayed with the examination information 102 for the examinations to be performed for the chronic diseases and the complications. The GUI control unit 41 of the client terminal 11 receives the distribution of the display screen edited as described above, and the distributed screen is displayed on the screen of the display unit 36 instead of the initial screen 71 initially displayed.

As described above, in the medical care support system 110 and the medical care support device 112 of the present embodiment, in a case where a patient suffers from a chronic disease, proposals are made not only for the affected chronic diseases but also for complications that are likely to occur, and necessary examinations and treatments can be promptly proposed to patients.

In the present embodiment, by centrally managing the learning unit 125 and the prediction execution unit 126 on the external server 113, for example, in a case where the teacher data is collected as big data on the web, it is possible to guarantee the prediction accuracy of the medical care support devices 112 of all the medical facilities constituting the medical care support system 110 by selecting only teacher data that guarantees accuracy (reliability), or by executing a version update of the trained model after verifying the accuracy of the updated trained model.

### Third Embodiment

In the first and second embodiments described above, the display screen generation unit proposes a medical care practice based on the prediction result predicted by the prediction execution unit, but the present invention is not limited thereto, and in a case where the user performs a different medical care practice with respect to the proposed medical care practice, the display screen generation unit may propose a medical care practice that reflects a prediction correction result corrected from the medical care practice performed by the user. The configuration of the entire medical care support system is the same as that of the medical care support system 110 of the second embodiment, and the description thereof will be omitted. Further, the same devices or configurations as those of the first and second embodiments are designated by the same reference numerals and the description thereof will be omitted.

As shown in FIG. 17, the CPU 51 of the medical care support device 132 functions as the request reception unit 61, a display screen generation unit 135, a prediction correction unit 136, a user correction storage unit 137, the external server communication unit 122, and the like in cooperation with the memory 52.

The display screen generation unit 135 generates or edits various display screens in the same manner as the display screen generation unit 121 of the second embodiment. The display screen generation unit 135 proposes a medical care practice to the client terminal 11 based on a prediction correction result in which a prediction correction unit 136, which will be described later, has corrected the prediction result. The display screen generation unit 135 generates or edits XML data representing the display screen by using the prediction correction result of the chronic disease, complications, and the like for which the prediction correction unit 136 has corrected the prediction result, and transmits the XML data to the client terminal 11. The operation in which the display screen generation unit 135 makes a proposal to the client terminal 11 based on the prediction correction result in which the prediction correction unit 136 has corrected the prediction result constitutes a medical care practice proposal step.

For example, in a case where the medical staff who is the user performs a different medical care practice with respect to the medical care practice proposed by the display screen generation unit 135 among various processing requests from the client terminal 11 received by the request reception unit 61, the user correction storage unit 137 extracts the medical care practice (addition, deletion, or correction of the medical care practice) corrected by the user as the user correction content.

For example, as a proposal of the display screen generation unit 135, in a case where information on the drug to be administered for diabetes is displayed and the user administers a different type of drug, the user operates the client terminal 11 to input that a different type of drug has been administered. The user correction storage unit 137 extracts as the user correction content that different types of drugs have been administered from the various processing requests received by the request reception unit 61 from the client terminal 11. The user correction storage unit 137 accumulates the medical care practice extracted as the user correction content.

The prediction correction unit 136 receives the prediction result in which the prediction execution unit 126 predicts a chronic disease, complications, and the like from the medical care information via the medical care support device communication unit 127, the network 114, and the external server communication unit 122. The prediction correction unit 136 outputs a prediction correction result obtained by correcting the prediction result using the user correction content accumulated by the user correction storage unit 137.

The medical care support system configured as described above operates as follows. Note that, the process is the same as in the first embodiment from the time when the medical staff logs in to the medical care support system 110 using the client terminal 11 until the request reception unit 61 receives the distribution request for the display screen, and the description thereof will be omitted.

In a case where the user has not yet performed a different medical care practice with respect to the medical care practice proposed on the layout display screen 101, as in the second embodiment, the display screen generation unit 135 proposes a medical care practice to the client terminal 11 based on the prediction result output by the prediction execution unit 126.

In a case where the medical staff who is the user performs a different medical care practice with respect to the medical care practice proposed by the display screen generation unit 135, the user correction storage unit 137 accumulates the medical care practice performed by the user as a user correction content. The prediction correction unit 136 outputs a prediction correction result obtained by correcting the prediction result using the user correction content accumulated by the user correction storage unit 137.

Next, the display screen generation unit 135 proposes a medical care practice to the client terminal 11 based on the prediction correction result. That is, the display screen generation unit 135 edits the display screen in which the examination information reflecting the user correction content is superimposed and displayed on the layout display screen 101 for the examinations to be performed for the chronic disease and complications. The GUI control unit 41 of the client terminal 11 receives the distribution of the display screen edited as described above, and the distributed screen is displayed on the screen of the display unit 36 instead of the initial screen 71 initially displayed.

As described above, in the medical care support system and the medical care support device 132 of the present embodiment, in a case where a patient suffers from a chronic disease, proposals are made not only for the affected chronic diseases but also for complications that are likely to occur, and necessary examinations and treatments can be promptly proposed to patients.

On the other hand, standards for examinations or medications may be set independently by medical facilities. In the present embodiment, a user correction content corrected by the user for the medical care practice proposed by the medical care support device 132 is accumulated, and the medical facility to which the user belongs proposes a medical care practice that reflects the user correction content. Therefore, even though the medical facility has its own standards, it is possible to propose medical care practices that can be handled.

As a modification example of the third embodiment, in addition to proposing the medical care practice reflecting the user correction content accumulated by the user correction storage unit 137 in the medical facility, a new trained model may be generated by performing machine learning using the user correction content accumulated by the user correction storage unit 137 as teacher data, and the trained model used for the prediction execution unit may be updated.

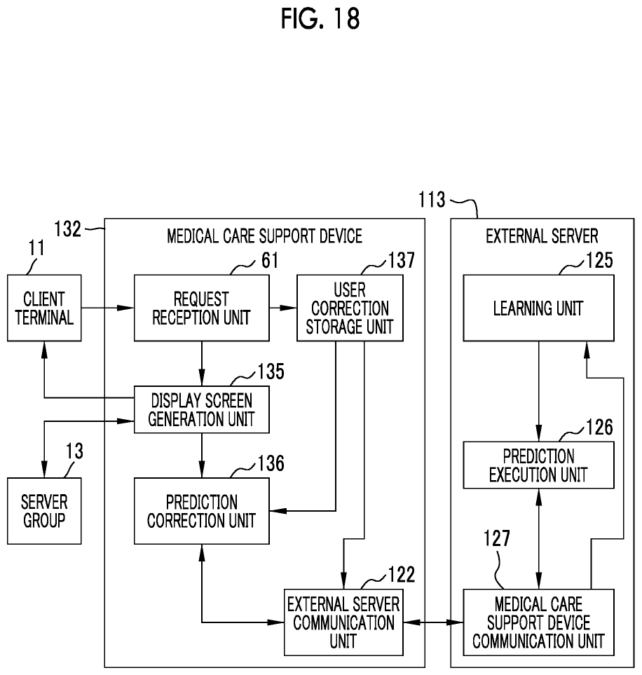

In a modification example shown in FIG. 18, the user correction storage unit 137 transmits the user correction content to the learning unit 125 via the external server communication unit 122, the network 114, and the medical care support device communication unit 127. The learning unit 125 generates a new trained model by performing machine learning using the user correction content as teacher data. The trained model generated by the learning unit 125 is output to the prediction execution unit 126. In a case where a trained model is generated, as in the second embodiment, the trained model used for the prediction execution unit 126 may be constantly updated to the latest version, or the trained model may be updated periodically, such as the case of updating the version.

In the medical facilities, some hospitals boast the leading treatment records for certain diseases, and some hospitals provide advanced diagnosis or treatment. By selecting the user correction contents acquired in such a medical facility and performing machine learning, or by weighting the user correction contents acquired in the medical facility as described above and performing machine learning, the prediction accuracy of the trained model can be further improved.

### Fourth Embodiment

In each of the above embodiments, the proposal made by the display screen generation unit based on the prediction result is only the display of examination and treatment information, but the present invention is not limited thereto. As a proposal made by the medical care support device, a proposal time for performing an examination or treatment for a chronic disease and complications may be determined, and a patient who has not yet undergone the examination or treatment at the proposal time may be displayed on the terminal device.

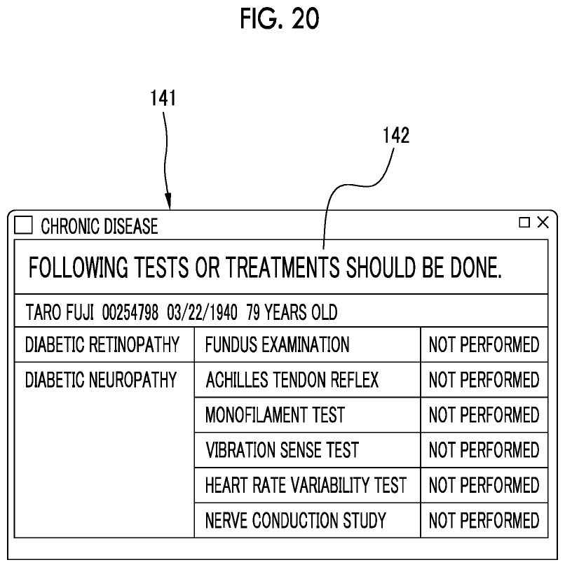

The processing of the proposal performed by the display screen generation unit in the present embodiment will be described with reference to the flowchart shown in FIG. 19 and the explanatory diagram shown in FIG. 20. The configuration other than the display screen generation unit is the same as that of each of the above embodiments, and the description thereof will be omitted. First, the display screen generation unit receives the prediction result predicted for a chronic disease, complications, and the like from the medical care information acquired by the prediction execution unit (S101).

Then, the display screen generation unit determines a proposal time for performing the examination or treatment for the chronic disease and complications of the target patient from the prediction result (S102). For example, in a case where a patient suffers from diabetes and also has diabetic retinopathy, there is a fundus examination or the like as an examination for diabetic retinopathy, and a prediction result of the examination frequency of once a year is output (see FIG. 8). Then, the display screen generation unit reads out the date of the previous examination from the medical care information, and determines, for example, a date within one year from the previous examination as a proposal time for performing the examination. In a case where the prediction result is corrected using the user correction content as in the third embodiment, the proposal time for performing the examination is determined from the prediction correction result reflecting the user correction content.

Next, the display screen generation unit monitors whether or not the current date is the proposal time (S103). Then, in a case where the current date is the proposal time (Y in S103), the display screen generation unit confirms whether or not the target patient is undergoing an examination or treatment for a chronic disease and complications (S104). In a case where the patient has not been examined or treated (N in S104), the patient who has not been examined or treated is displayed on the display unit 36 of the client terminal 11 (S105) as shown in a display screen 141 shown in FIG. 20. The display screen 141 includes that six types of tests have not been performed on the target patient “Taro Fuji”, a display 142 that prompts the examination or treatment, and the like.

As described above, even though the period from the previous examination or treatment is long, the display is made at the proposal time for performing the examination, so that the user or patient does not forget to receive the examination or treatment.

In each of the above embodiments, as a proposal for a medical care practice, an example of medication as a treatment for a chronic disease or a complication that is likely to occur with a morbidity of the chronic disease is given. However, the present invention is not limited thereto, and other treatments such as surgery and physical therapy may be proposed.

In each of the above embodiments, a chronic disease is exemplified as a specific disease, and complications that are likely to occur with the morbidity of the chronic disease are exemplified. However, the present invention is not limited thereto, a disease that causes another disease by being affected is a specific disease, and a disease that is likely to occur with the morbidity of the specific disease is a comorbidity.

In each of the above embodiments, the prediction execution unit acquires medical care information of the patient and extracts a chronic disease affecting the patient from the acquired medical care information, but the present invention is not limited thereto, and the chronic disease affecting the patient may be predicted from the patient's symptoms in the acquired medical care information.

In each of the above embodiments, hardware structures of the processing units that execute various processes such as the GUI control unit 41, the request issuing unit 42, the request reception unit 61, the display screen generation unit 62, the display screen generation unit 121, the display screen generation unit 135, the prediction execution unit 63, the prediction execution unit 126, the external server communication unit 122, the learning unit 125, the medical care support device communication unit 127, and the prediction correction unit 136 are various processors as shown below. The various processors include a central processing unit (CPU) as a general-purpose processor functioning as various processing units by executing software (program), a programmable logic device (PLD) as a processor of which the circuit configuration can be changed after manufacturing such as a field programmable gate array (FPGA), a dedicated electrical circuit as a processor having a circuit configuration designed exclusively for executing various kinds of processing, and a graphical processing unit (GPU), and the like.

One processing unit may be configured by one of various processors, or may be configured by a combination of the same or different kinds of two or more processors (for example, a combination of a plurality of FPGAs, a combination of a CPU and an FPGA, or a combination of a GPU and a CPU). In addition, a plurality of processing units may be configured by one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements. According to another aspect of the present invention, there is provided a medical care support device comprising a processor configured to acquire operation histories in a case where a plurality of terminal devices installed in a plurality of medical facilities are operated, from the terminal devices, predict next operation candidates in a case where the terminal devices are input and operated by using a trained model generated by learning the acquired operation histories by an external server installed outside the medical facilities, and make proposals to the terminal devices from the next operation candidates.

It goes without saying that the present invention is not limited to the above-described embodiment, and various configurations can be adopted as long as the gist of the present invention is not deviated. Further, the present invention is employed to a storage medium for storing the program in addition to the program.

From the above description, the medical care support device according to the following Additional Items 1 and 2 can be grasped.

[Additional Item 1]

A medical care support device comprising a processor, in which the processor is configured to acquire medical care information of a patient from a terminal device or a server installed in a medical facility, acquire a specific disease affecting the patient from the acquired medical care information, and outputs the specific disease and a comorbidity that is likely to occur with a morbidity of the specific disease as a prediction result, and propose medical care practices for both the specific disease and the predicted comorbidity.

[Additional Item 2]

A medical care support device comprising a processor, in which the processor is configured to acquire medical care information of a patient from a terminal device or a server installed in a medical facility, and propose medical care practices for both a specific disease and a comorbidity that is likely to occur with a morbidity of the specific disease from the acquired medical care information by using a prediction result predicted by a prediction execution processor installed outside the medical facility, the prediction result including the specific disease affecting the patient and the comorbidity.

### EXPLANATION OF REFERENCES

10, 110: medical care support system
11: client terminal
12, 112, 132: medical care support device
13: server group
14, 114: network
21: electronic medical record server
21A: medical record database
22: image server
22A: image database
23: report server
23A: report database
31, 51: central processing unit (CPU)
32, 52: memory
33, 53: storage
34, 54: communication unit
35, 55: connection circuit
36: display unit
37: operation unit
39, 59: operation program
41: graphical user interface (GUI) control unit
42: request issuing unit
61: request reception unit
62, 121, 135: display screen generation unit
63, 126: prediction execution unit
71: initial screen
72: schedule display field
73: mail display field
74: list display field
78, 79: scroll bar
81: clinical flow screen
101: layout display screen
102: examination information
103, 142: display
113: external server
122: external server communication unit
125: learning unit
127: medical care support device communication unit
136: prediction correction unit
137: user correction storage unit
141: display screen
A1, A2, B1: doctor
G1, G2, G19: group
N1: technician

What is claimed is:

1. A medical care support device comprising:
a processor configured to:
acquire medical care information of a patient from a terminal device or a server installed in a medical facility;
acquire a specific disease affecting the patient from the acquired medical care information, and output the specific disease and a comorbidity that is likely to occur with a morbidity of the specific disease as a prediction result; and
propose medical care practices for both the predicted specific disease and the comorbidity.

2. A medical care support device comprising:
a processor configured to:
acquire medical care information of a patient from a terminal device or a server installed in a medical facility; and
propose medical care practices for both a specific disease and a comorbidity that is likely to occur with a morbidity of the specific disease from the acquired medical care information by using a prediction result predicted by a prediction execution processor installed outside the medical facility, the prediction result including the specific disease affecting the patient and the comorbidity.

3. The medical care support device according to claim 1,
wherein the processor includes, as the prediction result, an examination or treatment to be performed for the specific disease and the comorbidity, and
displays, as the proposal, the examination or treatment on the terminal device.

4. The medical care support device according to claim 2,
wherein the prediction execution processor includes, as the prediction result, an examination or treatment to be performed for the specific disease and the comorbidity, and
the processor displays, as the proposal, the examination or treatment on the terminal device.

5. The medical care support device according to claim 1,
wherein the processor includes, as the prediction result, a proposal time for performing an examination or treatment for the specific disease and the comorbidity, and
displays, as the proposal, a patient who has not yet undergone the examination or treatment at the proposal time on the terminal device.

6. The medical care support device according to claim 2,
wherein the prediction execution processor includes, as the prediction result, a proposal time for performing an examination or treatment for the specific disease and the comorbidity, and
the processor displays, as the proposal, a patient who has not yet undergone the examination or treatment at the proposal time on the terminal device.

7. The medical care support device according to claim 1, wherein the processor predicts the specific disease affecting the patient from the medical care information, and predicts the comorbidity from the predicted specific disease.

8. The medical care support device according to claim 2, wherein the prediction execution processor predicts the specific disease affecting the patient from the medical care information, and predicts the comorbidity from the predicted specific disease.

9. The medical care support device according to claim 1, further comprising:
a user correction storage that, in a case where a user performs a different medical care practice with respect to the proposed medical care practices, accumulates the medical care practice performed by the user as a user correction content,
wherein the processor outputs a prediction correction result obtained by correcting the prediction result using the accumulated user correction content, and
proposes a medical care practice in which the prediction correction result is reflected.

10. The medical care support device according to claim 2, further comprising:
a user correction storage that, in a case where a user performs a different medical care practice with respect to the proposed medical care practices, accumulates the medical care practice performed by the user as a user correction content,
wherein the processor outputs a prediction correction result obtained by correcting the prediction result using the accumulated user correction content, and
proposes a medical care practice in which the prediction correction result is reflected.

11. The medical care support device according to claim 9,
wherein the processor outputs a comorbidity that is likely to occur for a predetermined specific disease and medical care practices for both the specific disease and the comorbidity by a trained model that outputs the comorbidity and the medical care practices, and
updates the trained model as a new trained model by performing machine learning using the accumulated user correction content as teacher data.

12. The medical care support device according to claim 10,
wherein the prediction execution processor outputs a comorbidity that is likely to occur for a predetermined specific disease and medical care practices for both the specific disease and the comorbidity by a trained model that outputs the comorbidity and the medical care practices, and
updates the trained model as a new trained model by performing machine learning using the accumulated user correction content as teacher data.

13. The medical care support device according to claim 1, wherein the specific disease is a chronic disease, and the comorbidity is a complication that is likely to occur with a morbidity of the chronic disease.

14. The medical care support device according to claim 1, wherein the medical care practice includes the examination for the specific disease or the comorbidity.

15. The medical care support device according to claim 1, wherein the medical care practice includes medication for the patient with the specific disease.

16. A medical care support system comprising the medical care support device according to claim 1, a terminal device, and an external server.

17. An operation method of a medical care support device, the method comprising:
acquiring medical care information of a patient from a terminal device or a server installed in a medical facility;
extracting a specific disease affecting the patient from the acquired medical care information, and outputting the specific disease and a comorbidity that is likely to occur with a morbidity of the specific disease as a prediction result; and
proposing medical care practices for both the predicted specific disease and the comorbidity.

18. An operation method of a medical care support device, the method comprising:
acquiring medical care information of a patient from a terminal device or a server installed in a medical facility; and
proposing medical care practices for both a specific disease and a comorbidity that is likely to occur with a morbidity of the specific disease from the acquired medical care information by using a prediction result, predicted by a prediction execution processor installed outside the medical facility, the prediction result including the specific disease affecting the patient and the comorbidity.

19. A non-transitory computer readable recording medium storing an operation program causing the computer to execute the operation method of a medical care support device according to claim 17.

20. A non-transitory computer readable recording medium storing an operation program causing the computer to execute the operation method of a medical care support device according to claim 18.
